(19) United States
(12) Patent Application Publication
(10) Pub. No.: US 2023/0050095 A1
(43) Pub. Date: Feb. 16, 2023

(54) CHAIR CAPABLE OF PROVIDING SOMATOSENSORY VIBRATION BASED ON EXTERNAL SIGNAL

(71) Applicant: IDISPLAY TECHNOLOGY CO., LTD., New Taipei City (TW)

(72) Inventor: Hank Ching-Tsung CHEN, New Taipei City (TW)

(21) Appl. No.: 17/399,614

(22) Filed: Aug. 11, 2021

(51) Int. Cl. A61M 21/00; G08B 7/06; H04R 3/04; A61G 15/00

(52) U.S. Cl. CPC A61M 21/00; G08B 7/06; H04R 3/04; A61G 15/007; A61M 2021/0022; A61M 2021/0027

(57) ABSTRACT

A chair capable of providing somatosensory vibration based on an external signal comprises a body, a plurality of vibrators and a vibration controller. The body comprises a framework and a plurality of mesh fabrics disposed on the framework. The vibrators are hung on the framework through at least two connectors and are arranged corresponding to one of the mesh fabrics. Each of the plurality of vibrators is provided with a vibration surface directly contacting one of the mesh fabrics. Each of the plurality of vibrators operates based on at least one vibration starting signal. The vibration controller is disposed on the body and is in data connection with the vibrators. The vibration controller generates at least one vibration starting signal based on a received external signal, and provides at least one vibration starting signal to each of the plurality of vibrators in a wired or wireless manner.

### FIELD OF THE INVENTION

The invention relates to a chair capable of providing somatosensory vibration, in particular to a chair capable of providing somatosensory vibration based on an external signal.

### BACKGROUND OF THE INVENTION

A conventional chair having a vibrator is disclosed in U.S. Pat. No. 7,744,153. In particular, the invention discloses a chair comprising a massage device disposed on a backrest portion of the chair, the massage device being activated after being operated to vibrate the chair, thereby providing a massage function to a user. This type of vibrator operates when the user starts the vibrator, then the vibrator receives power to start vibrating. Once the user operates the vibrator again, the power of the vibrator would be cut off, so that the vibrator stops vibrating. The conventional vibrator is controlled by power conduction so that an external device is unable to be used as a control source, which means the conventional chair does not have the function of providing vibration based on an external signal.

In view of this, current chair manufacturers have developed chairs capable of receiving external signals in order to overcome the aforementioned problems. These chairs are commonly referred to as 4D chairs or immersion chairs. These chairs are connected to external devices and use the external devices as signal control sources. The chairs begin to vibrate after receiving external signals from the external devices. However, the vibrator arrangements of these chairs currently on the market include that: the vibrator is directly locked on the chair or embedded in the seat cushion or the seat backrest.

Though the type of the chairs with vibrator directly locked on is the simplest form of assembly, as the chair is provided with movable elements or parts which are not tightly assembled, the vibration of the vibrator easily causes the noise of the chair. Even more, the kinetic energy of the vibrator is transmitted to the ground through the chair to affect others.

Furthermore, take the embedded vibrator for description. As the vibrator is disposed in the seat cushion, the vibration surface thereof is combined with the cushion body to transfer kinetic energy, which results the cushion body deformed or sunk by the gravity of the vibrator. The user would easily feel foreign objects when sitting on it. In addition, the existing cushion material has soft characteristics, such as foam cotton and the like to provide comfortable sitting feeling for a user, which means the vibration energy of the vibrator is easily absorbed by the cushion material. As a plurality of vibrators is disposed on the same plane, a user is unable to specifically feel the directionality of a vibration source due to the influence of pad material.

### SUMMARY OF THE INVENTION

The invention mainly aims to solve the problem that conventional chairs cannot be controlled based on an external signal.

Another object of the invention is to solve the problem that the provided manner of a conventional vibrator is unable to provide a good body feeling experience.

In order to achieve the object, the invention provides a chair capable of providing somatosensory vibration based on an external signal. The chair comprises a body, a plurality of vibrators, and a vibration controller. The body comprises a framework and a plurality of mesh fabrics disposed on the framework. The plurality of vibrators is hung on the framework through at least two connectors and arranged corresponding to one of the mesh fabrics. Each of the plurality of vibrators is provided with a vibration surface directly contacting one of the mesh fabrics. Each of the plurality of vibrators operates based on at least one vibration starting signal. The vibration controller is disposed on the body and is data connected with the plurality of vibrators. The vibration controller generates the at least one vibration starting signal based on a received external signal, and the vibration controller provides the at least one vibration starting signal to each of the plurality of vibrators in a wired or wireless manner.

In one embodiment, the framework is formed with a plurality of fixing hooks for respectively hanging one of the connectors.

In one embodiment, each of the connectors is an elastic band.

In one embodiment, each of the plurality of vibrators comprises a damping body and at least one vibration member disposed on the damping body.

In one embodiment, one of the vibrators includes a plurality of vibration members, and the plurality of vibration members are arranged at intervals.

In one embodiment, the damping body comprises a first surface facing one of the mesh fabrics as the vibration surface, a second surface opposite to the first surface, and at least one receiving groove formed in the second surface for the vibration member to be provided therein.

In one embodiment, one of the plurality of vibrators comprises an auxiliary plate disposed on the second surface, and a positioning hole disposed on the auxiliary plate and assembled with a positioning member on the framework, and the auxiliary plate comprises a buffer column, the positioning hole being disposed on the buffer column.

In one embodiment, one of the plurality of vibrators comprises an auxiliary plate disposed on the second surface, and a positioning hole disposed on the auxiliary plate and assembled with a positioning member on the framework.

In one embodiment, the mesh fabrics are formed with two mesh covers sleeved on the framework, and the plurality of vibrators are respectively disposed on the two mesh covers.

In an embodiment, the vibrators are in data connection with the vibration controller through a plurality of transmission lines, and the plurality of transmission lines are respectively accommodated in the two mesh covers.

In one embodiment, the body is divided into a backrest portion and a seat portion, at least one of the plurality of vibrators is disposed on the backrest portion, and one of the plurality of vibrators is disposed on the seat portion.

In one embodiment, one, disposed on the seat portion, of the plurality of vibrators is located at an end of the seat portion opposite to the backrest portion.

In one embodiment, the chair comprises an auxiliary vibrator in data connection with the vibration controller, and the mesh fabrics define a bearing surface on which the auxiliary vibrator is provided.

In one embodiment, the body is divided into a backrest portion and a seat portion, and the auxiliary vibrator is disposed on the backrest portion.

In one embodiment, the vibration controller obtains the external signal from a multimedia provision device in a wired or wireless manner.

In one embodiment, the external signal is an audio signal, and the vibration controller filters the audio signal and takes a low frequency part of the audio signal to generate the vibration starting signal.

In one embodiment, the low frequency part is a part of the audio signal below 250 Hz.

In one embodiment, the external signal is a digital signal, the vibration controller decodes the external signal to generate an audio signal, and generates the vibration starting signals based on the audio signal.

In one embodiment, a low frequency part of the audio signal is taken to generate the vibration starting signals after the audio signal is filtered.

Through the implementation of the invention, compared with the conventional application, the invention includes: the vibration controller receives the external signal to provide the vibration starting signals to the vibrators based on the external signal, so that the vibrators vibrate based on the vibration starting signals. On the other hand, the vibrators are not embedded in the cushion body any more, the vibrators are closer to a user when vibrating. The problem that the user cannot really feel the vibration due to the isolation of the cushion body in the prior art is solved. Meanwhile, the vibrators of the invention solve the problem that since the cushion body of the chair is thickened, the vibrators must vibrate greatly to be unfavorable to the chair in order to solve the foreign object feeling of a user in a conventional device.

### BRIEF DESCRIPTION OF THE DRAWINGS

FIG. 1 is a schematic structural perspective view of an embodiment of the invention.

FIG. 2 is a schematic structural perspective view of another aspect of an embodiment of the invention.

FIG. 3 is a schematic view of a control mode of an embodiment of the invention.

FIG. 4 is a schematic structural perspective view of partial elements of an embodiment of the invention.

FIG. 5 is a schematic structural view of partial elements of another aspect of an embodiment of the invention.

FIG. 6 is a schematic structural exploded view of partial elements of an embodiment of the invention.

FIG. 7 is a schematic structural exploded view of partial elements of an embodiment of the invention.

FIG. 8 is a schematic view of a mesh sleeve arrangement of an embodiment of the invention.

FIG. 9 is a schematic view of an auxiliary vibrator arrangement of an embodiment of the invention.

### DETAILED DESCRIPTION OF THE PREFERRED EMBODIMENTS

The detailed description and technical contents of the invention are described with reference to the drawings as follows.

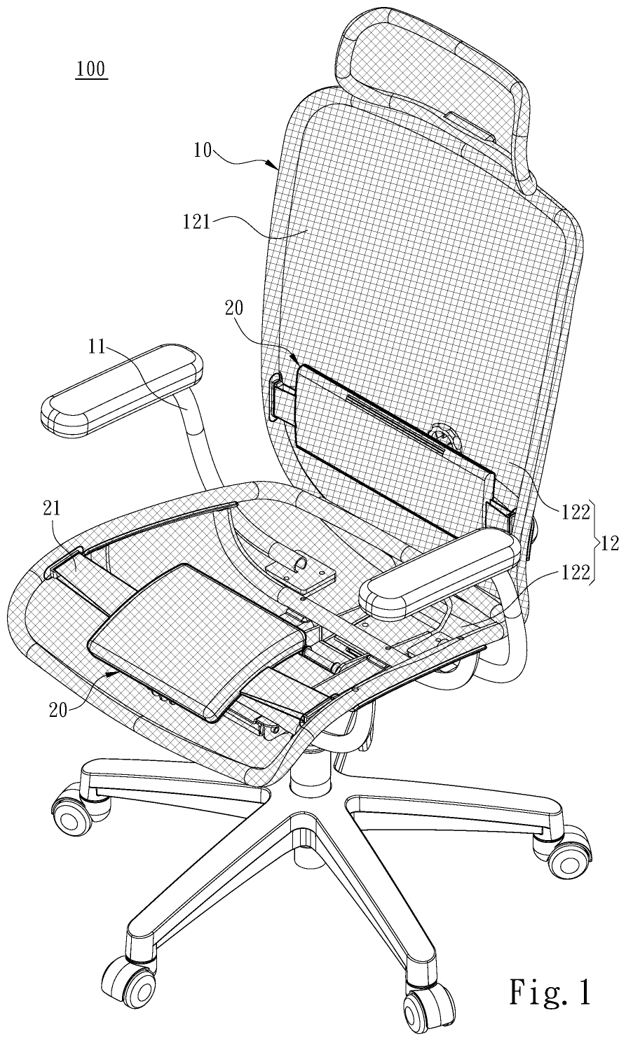

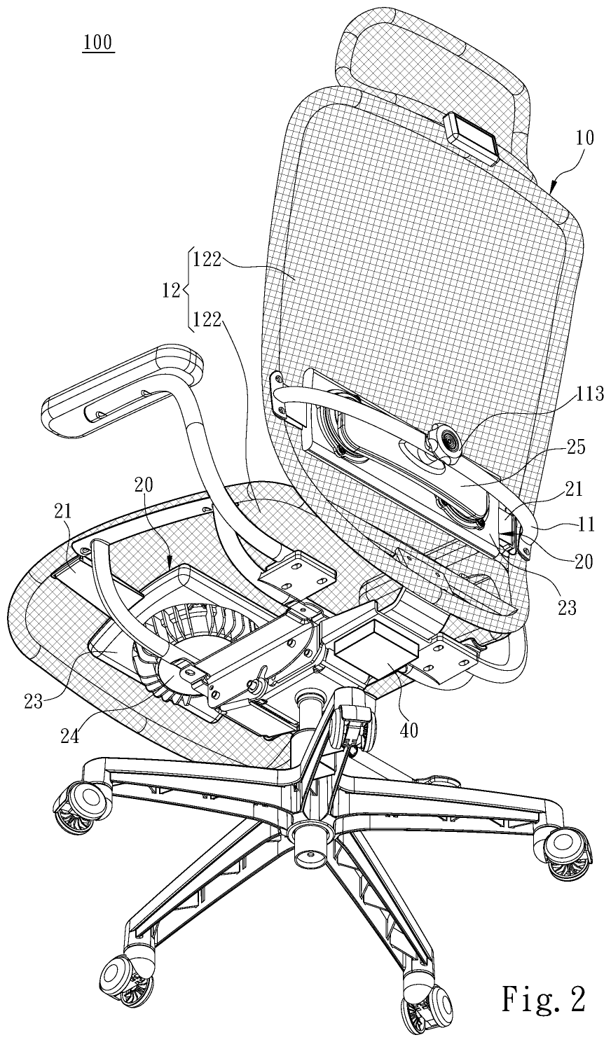

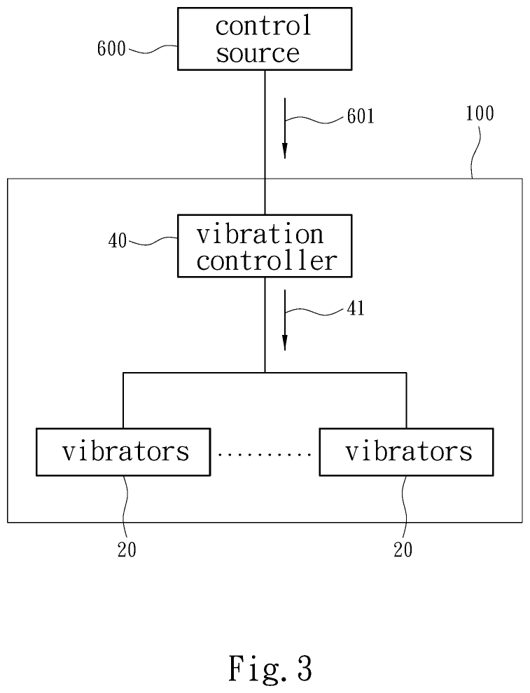

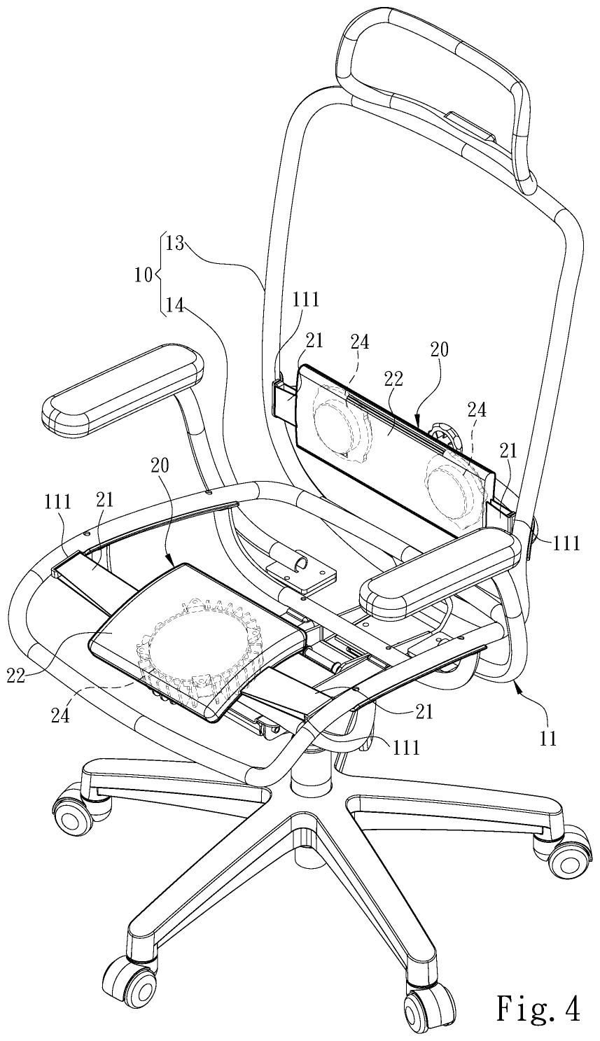

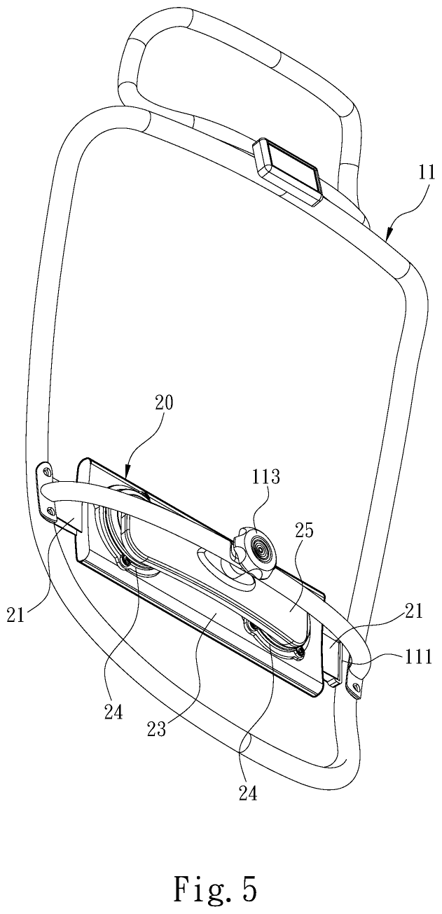

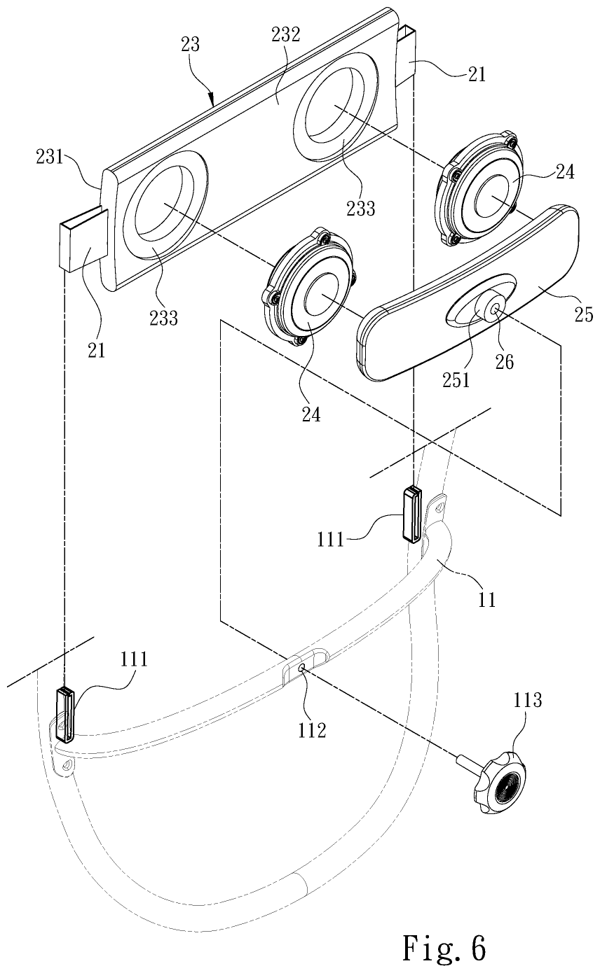

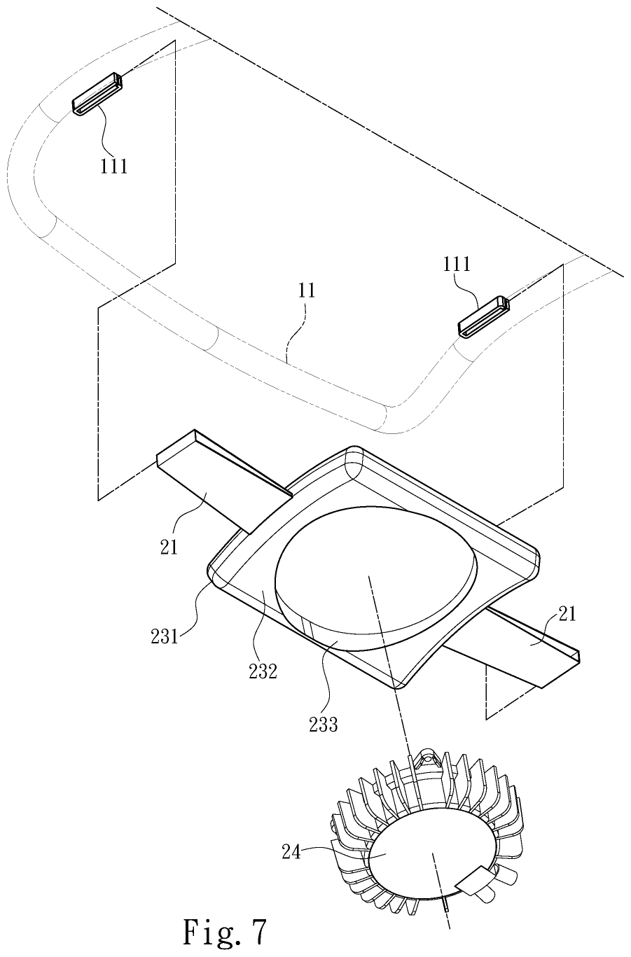

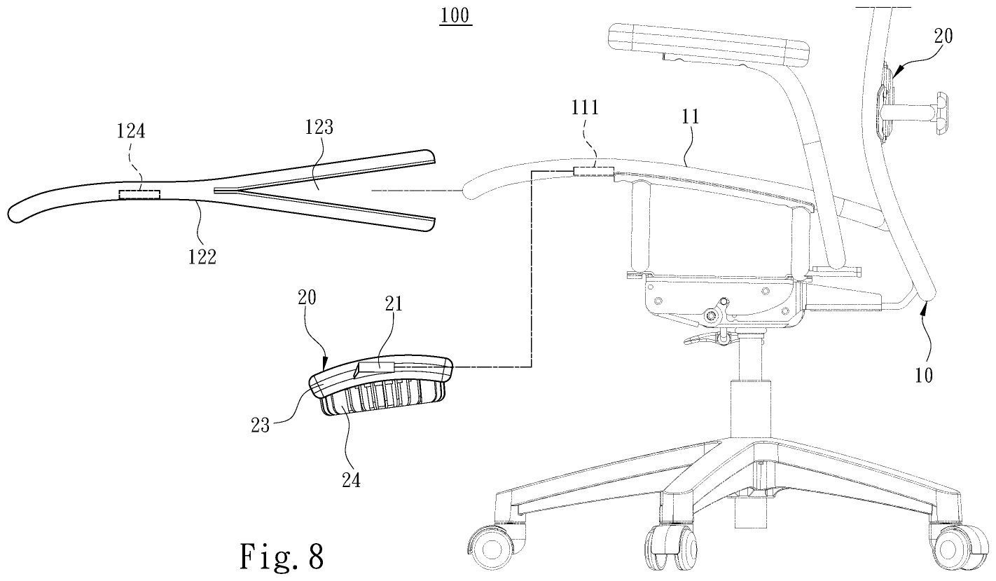

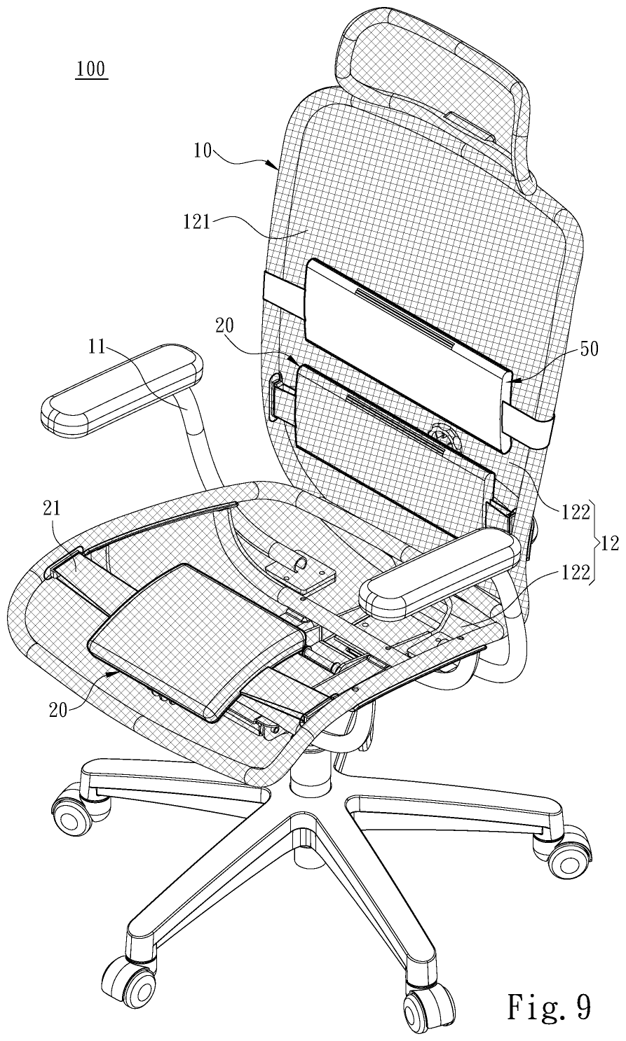

With reference to FIG. 1 to FIG. 9, the invention provides a chair 100 capable of providing somatosensory vibration based on an external signal. The invention comprises a body 10, a plurality of vibrators 20, and a vibration controller 40. The body 10 for sitting comprises a framework 11 and a plurality of mesh fabrics 12 disposed on the framework 11. The vibrators 20 are hung on the framework 11 through at least two connectors 21 and are arranged corresponding to one of the mesh fabrics 12. In detail, a bearing surface 121 on the framework 11 for bearing is formed by the mesh fabrics 12. The mesh fabrics 12 include a plurality of meshes on the side which is facing the sitter (such as the bearing surface 121). The diameter of the meshes is larger than the knitting of the mesh fabrics 12. In one embodiment, the mesh fabrics 12 are one-piece knitted by a knitting technique. The mesh fabrics 12 are able to be implemented in a single, double, or multi-layer construction. In a multi-layer construction, the mesh fabrics 12 are able to be implemented in a spacer fabric form commonly known as a sandwich mesh. The mesh on a surface where the spacer fabric is located is also formed by knitting techniques. In addition, as the mesh fabrics 12 are implemented in a double-layer or multi-layer construction, the mesh fabrics 12 are able to be one of the following states when viewed from one side: full penetration, half penetration, no penetration. The vibrators 20 are provided at the lower side of bearing surface 121 without affecting the seating function of the body 10. The vibrators 20 are provided with a vibration surface 22, and the vibration surface 22 is directly contacted with one of the mesh fabrics 12. As each of the vibrators 20 operates based on at least one vibration starting signal 41, the vibration surface 22 directly vibrates the corresponding mesh fabrics 12. The vibration controller 40 is disposed on the body 10 and is data connected with the vibrators 20. The vibration controller 40 receives an external signal 601, which is from an external control source 600 in data connection with the vibration controller 40. The vibration controller 40 generates the at least one vibration starting signal 41 based on the external signal 601 and transmits the at least one vibration starting signal 41 to the vibrators 20. The vibration controller 40 transmits the at least one vibration starting signal 41 to the vibrators 20 via wireless transmission, such as Bluetooth, or via a plurality of transmission lines (not shown) connecting the vibration controller 40 and the vibrators 20 in a wired manner. The transmission lines could be accommodated in the mesh fabrics 12 during implementation so as to prevent the transmission lines from scattering outside the body 10.

Next, the implementation of the chair 100 of the invention is described. Assume initially that the vibration controller 40 does not receive the external signal 601 and is not driven. Once the vibration controller 40 receives the external signal 601 from the external control source 60, as the external signal 601 is an audio signal, the vibration controller 40 filters the external signal 601 and then generates the at least one vibration starting signal 41 based on the filtered external signal 601. Once the external signal 601 is a digital signal, the vibration controller 40 first decodes the external signal 601 and then generates the at least one vibration starting signal 41 based on the decoded external signal 601. Further, as the vibrators 20 respectively receive the at least one vibration starting signal 41, the vibrators 20 respectively operate at different amplitude levels, vibration frequencies, or vibration durations based on the received at least one vibration starting signal 41.

Accordingly, the invention receives the external signal 601 from the vibration controller 40 and the vibration controller 40 provides the start signal 41 to the vibrators 20 based on the external signal 601, so that the vibrators 20 vibrate based on the vibration starting signal 41. On the other hand, the invention provides the vibrators 20 closer to a user to improve the conventional embedding manner thereof the vibrators barely transmit a good vibration effect due to the isolation of the cushion body. Meanwhile, since the vibrators 20 of the invention are not embedded in the cushion body, the vibrators 20 vibrate with lower vibration amplitude than the conventional ones, and the influence caused when the conventional chair shakes along with the vibrators is improved. In addition, as the vibrators 20 are not locked on the chair 100, the vibrators 20 reduce collision with the chair 100 and abnormal sounds generated during collision while vibrating.

In one embodiment, a plurality of fixing hooks 111 is formed on the framework 11. The fixing hooks 111 are respectively provided for one of the connectors 21, so that the vibrators 20 are suspended on the framework 11. The vibration surface 22 of each of the vibrators 20 is not in contact with the framework 11, thereby reducing the transmission of vibration energy to the framework 11 when the vibrators 20 vibrate. In addition, the connectors 21 of the invention could respectively be an elastic band which includes the function of absorbing energy to reduce vibration of the framework 11 when the vibrators 20 vibrate.

Furthermore, the vibrators 20 are respectively provided with a damping body 23 and at least one vibration member 24 disposed on the damping body 23, wherein the damping body 23 is made of a soft material such as rubber or silica gel and is disposed on the mesh fabric 12 through hot melting and the like. The damping body 23 deforms when a user sits on the body 10, so that the discomfort caused by the user pushing against the vibration member 24 is reduced. In one embodiment, the damping body 23 includes a first surface 231 facing one of the mesh fabrics 12, a second surface 232 opposite the first surface 231, and at least one receiving groove 233 formed in the second surface 232. The damping body 23 uses the first surface 231 as the vibration surface 22, and the receiving groove 233 is used for installing the vibration member 24. The vibration member 24 is brought close to the vibration surface 22 after assembly, which means that the vibration energy is surely transmitted to the mesh fabric 12 during vibration.

In addition, in one embodiment, one of the vibrators 20 includes an auxiliary plate 25 provided at the second surface 232 and a positioning hole 26 provided at the auxiliary plate 25. The framework 11 includes an opening 112 facing the positioning hole 26 and a positioning member 113 provided at the opening 112, and the positioning member 113 is assembled in the positioning hole 26 through the opening 112 to fix the vibrator 20. Further, in order to prevent the vibrator 20 from driving the framework 11 when the vibrator 20 vibrates, the auxiliary plate 25 is provided with a buffer column 251. The buffer column 251 is provided with the positioning hole 26, thereby allowing the auxiliary plate 25 and the buffer column 251 to absorb energy when the vibrator 20 vibrates as the vibrator 20 is fixed.

Furthermore, in one embodiment, the body 10 is divided into a backrest portion 13 and a seat portion 14. At least one of the vibrators 20 is disposed on the backrest portion 13 and one of the vibrators 20 is disposed on the seat portion 14. The at least one, disposed on the backrest portion 13, of the vibrators 20 is provided with the auxiliary plate 25 and the buffer column 251. Each of the at least one, disposed on the backrest portion 13, of the vibrators 20 is provided with a plurality of vibration members 24 which are arranged at intervals and could be respectively driven to provide different vibration directivity feelings when a user sits on the chair 100. In addition, the one of the vibrators 20 disposed on the seat portion 14 is provided at one end of the seat portion 14 opposite to the backrest portion.

In another embodiment, the mesh fabrics 12 are formed with two mesh covers 122. Each of two mesh covers 122 includes an opening 123. The opening is for one of the two mesh covers 122 to sleeve on and be selectively removable from the backrest portion 13 or the seat portion 14. The two mesh covers 122 are provided with a through hole 124 at a position corresponding to each of the fixing hooks 111. The through hole 124 is used for one of the fixing hooks 111 to pass through so that the vibrators 20 could be assembled with the fixing hooks 111.

In another aspect of the invention, the control source 600 could be a multimedia device, such as a computer, a television game, a palm game, a cell phone, etc. The vibration controller 40 could receive the external signal 601 transmitted by the multimedia device via Bluetooth, WIFI, or etc. in a wireless manner, or via a signal wire (not shown). In operation, the vibration controller 40 receives the audio signal, regarded as the external signal 601, from the multimedia device. Further, since the vibrators 20 of the invention are liable to generate abnormal sounds at a vibration frequency of an excessively high frequency, the vibration controller 40 filters the audio signal after receiving the audio signal. The vibration controller 40 captures a low frequency part of the audio signal. For example, a part of the audio signal frequency is lower than 250 Hz on which is based by the vibration controller 40 to generate the vibration starting signals 41. In addition, the multimedia device could also send the digital signal to the vibration controller 40. The digital signal is regarded as the external signal 601 by the vibration controller 40, and then the vibration controller 40 decodes the digital signal to generate an audio signal. After the vibration controller 40 filtered the audio signal, a low-frequency part of the audio signal is taken to generate the vibration starting signals 41.

In addition to the foregoing, the chair 100 of the invention includes an auxiliary vibrator 50 which is disposed on the bearing surface 121 and is in data connection with the vibration controller 40. The auxiliary vibrator 50 could be controlled in the same manner as the vibrators 20, which will not be described in detail herein. Further, the auxiliary vibrator 50 of the invention could be provided to the backrest portion 13 of the body 10 in a hanging manner so that the auxiliary vibrator 50 would correspond to a user's waist.

What is claimed is:

1. A chair capable of providing somatosensory vibration based on an external signal, comprising:
a body, comprising a framework and a plurality of mesh fabrics disposed on the framework;
a plurality of vibrators, respectively hung on the framework through at least two connectors and arranged corresponding to one of the mesh fabrics, each of the plurality of vibrators being provided with a vibration surface directly contacting one of the mesh fabrics, and each of the plurality of vibrators operating based on at least one vibration starting signal; and
a vibration controller, disposed on the body and in data connection with the plurality of vibrators, the vibration controller generating the at least one vibration starting signal based on a received external signal, and the vibration controller providing the at least one vibration starting signal to each of the plurality of vibrators in a wired or wireless manner.

2. The chair capable of providing somatosensory vibration based on the external signal according to claim 1, wherein the framework is formed with a plurality of fixing hooks for respectively hanging one of the connectors.

3. The chair capable of providing somatosensory vibration based on the external signal according to claim 1, wherein each of the connectors is an elastic band.

4. The chair capable of providing somatosensory vibration based on the external signal according to claim 1, wherein each of the plurality of vibrators comprises a damping body and at least one vibration member disposed on the damping body.

5. The chair capable of providing somatosensory vibration based on the external signal according to claim 4, wherein one of the plurality of vibrators comprises a plurality of vibration members, and the plurality of vibration members are arranged at intervals.

6. The chair capable of providing somatosensory vibration based on the external signal according to claim 4, wherein the damping body comprises a first surface facing one of the mesh fabrics as the vibration surface, a second surface opposite to the first surface, and at least one receiving groove formed in the second surface for the vibration member to be provided therein.

7. The chair capable of providing somatosensory vibration based on the external signal according to claim 6, wherein one of the plurality of vibrators comprises an auxiliary plate disposed on the second surface, a positioning hole disposed on the auxiliary plate and assembled with a positioning member on the framework, and the auxiliary plate comprises a buffer column, the positioning hole being disposed on the buffer column.

8. The chair capable of providing somatosensory vibration based on the external signal according to claim 6, wherein one of the plurality of vibrators comprises an auxiliary plate disposed on the second surface, and a positioning hole disposed on the auxiliary plate and assembled with a positioning member on the framework.

9. The chair capable of providing somatosensory vibration based on the external signal according to claim 1, wherein the mesh fabrics are formed with two mesh covers sleeved on the framework, and the plurality of vibrators are respectively disposed on the two mesh covers.

10. The chair capable of providing somatosensory vibration based on the external signal according to claim 9, wherein the plurality of vibrators are in data connection with the vibration controller through a plurality of transmission lines, and the plurality of transmission lines are respectively accommodated in the two mesh covers.

11. The chair capable of providing somatosensory vibration based on the external signal according to claim 1, wherein the body is divided into a backrest portion and a seat portion, at least one of the plurality of vibrators is disposed on the backrest portion, and one of the plurality of vibrators is disposed on the seat portion.

12. The chair capable of providing somatosensory vibration based on the external signal according to claim 11, wherein one, disposed on the seat portion, of the plurality of vibrators is located at an end of the seat portion opposite to the backrest portion.

13. The chair capable of providing somatosensory vibration based on the external signal according to claim 1, wherein the chair comprises an auxiliary vibrator in data connection with the vibration controller, and the mesh fabrics define a bearing surface on which the auxiliary vibrator is provided.

14. The chair capable of providing somatosensory vibration based on the external signal according to claim 13, wherein the body is divided into a backrest portion and a seat portion, and the auxiliary vibrator is disposed on the backrest portion.

15. The chair capable of providing somatosensory vibration based on the external signal according to claim 1, wherein the vibration controller obtains the external signal from a multimedia provision device in a wired or wireless manner.

16. The chair capable of providing somatosensory vibration based on the external signal according to claim 15, wherein the external signal is an audio signal, and the vibration controller filters the audio signal and takes a low frequency part of the audio signal to generate the vibration starting signal.

17. The chair capable of providing somatosensory vibration based on the external signal according to claim 16, wherein the low frequency part is a part of the audio signal below 250 Hz.

18. The chair capable of providing somatosensory vibration based on the external signal according to claim 15, wherein the external signal is a digital signal, the vibration controller decodes the external signal to generate an audio signal, and generates the vibration starting signals based on the audio signal.

19. The chair capable of providing somatosensory vibration based on the external signal according to claim 18, wherein a low frequency part of the audio signal is taken to generate the vibration starting signals after the audio signal is filtered.

20. The chair capable of providing somatosensory vibration based on the external signal according to claim 19, wherein the low frequency part is a part of the audio signal below 250 Hz.
